(19) United States
(12) Patent Application Publication
(10) Pub. No.: US 2023/0386035 A1
(43) Pub. Date: Nov. 30, 2023

(54) MEDICAL IMAGE PROCESSING SYSTEM

(71) Applicant: SHANGHAI UNITED IMAGING HEALTHCARE CO., LTD., Shanghai (CN)

(72) Inventors: CHUN-YU WANG, Shanghai (CN); CHUN-JING TANG, Shanghai (CN); YI-ZHE GENG, Shanghai (CN)

(21) Appl. No.: 18/117,442

(22) Filed: Mar. 5, 2023

(30) Foreign Application Priority Data
May 25, 2022 (CN) 2022105759060

(51) Int. Cl. G06T 7/00; G06T 7/33; G06T 7/73; G06V 10/25; G06V 10/22; G06T 3/40; G16H 15/00

(52) U.S. Cl. CPC G06T 7/0014; G06T 7/33; G06T 7/73; G06V 10/25; G06V 10/22; G06T 3/40; G16H 15/00; G06T 2207/30016; G06T 2207/30096; G06V 2201/03

(57) ABSTRACT

A medical image processing system includes a medical imaging device and a report generating device. The medical imaging device is configured to acquire a first image and a second image of a target part, and transmit the first image and the second image to the report generating device. The first image is used for depicting an anatomy structure of the target part, and the second image is used for depicting quantified parameter information of the target part. The report generating device is configured to identify a first region of interest (ROI) in the first image, and perform a registration for the first image and the second image to obtain a second ROI in the second image relevant to the first ROI in the first image, and further configured to generate a human readable report of the target part according to quantified parameter information corresponding to the second ROI in the second image.

### CROSS-REFERENCE TO RELATED APPLICATION

The present application claims the priority of Chinese Patent Application No. 202210575906.0, filed on May 25, 2022 and entitled “MEDICAL IMAGE PROCESSING SYSTEM”, which is hereby incorporated by reference in its entirety.

### TECHNICAL FIELD

The present disclosure relates to the field of computer technology, and more particularly, to a medical image processing system.

### BACKGROUND

With the development of magnetic resonance sequence technology, by magnetic resonance scanning for the liver, not only qualitative images may be obtained to provide plentiful contrast information for the anatomical structure of the liver to facilitate an identification of a lesion, but also multi-dimensional quantitative images may be acquired to provide multi-dimensional diagnostic information for a liver lesion.

### SUMMARY

The present disclosure provides a medical image processing system easy to operate, a medical image processing method, a computer apparatus, a non-transitory computer readable storage medium, and a computer program product.

In a first aspect, the present disclosure provides a medical image processing system. The system includes a medical imaging device and a report generating device.

The medical imaging device is configured to acquire a first image and a second image of a target part, and transmit the first image and the second image to the report generating device. The first image is used for depicting an anatomy structure of the target part, and the second image is used for depicting quantified parameter information of the target part.

The report generating device is configured to identify a first region of interest (ROI) in the first image, and perform a registration for the first image and the second image to obtain a second ROI in the second image; the second ROI in the second image is relevant to the first ROI in the first image.

The report generating device is further configured to generate a human readable report of the target part according to quantified parameter information corresponding to the second ROI in the second image.

In one of the embodiments, the report generating device is further configured to perform the registration for the first image and the second image to obtain a mapping relationship between the first image and the second image, and determine, according to the mapping relationship, an image region in the second image relevant to the first ROI to be the second ROI.

In one of the embodiments, the report generating device is further configured to determine first location information of the first ROI in the first image, determine, according to the mapping relationship, the second location information in the second image relevant to the first location information, and determine the second ROI in the second image according to the second location information.

In one of the embodiments, at least one second image is provided, and at least one second ROI in each of the at least one second image is provided. Each of the at least one second image is used for depicting quantified parameter information of the target part in different dimensions. The report generating device is further configured to generate the human readable report of the target part based on the first image, each of the at least one second image, and each quantified parameter information corresponding to each of the at least one second ROI.

In one of the embodiments, the report generating device is further configured to acquire an information reference value corresponding to each quantified parameter information, generate an information abnormality marker in a case that the quantified parameter information does not conform to the information reference value, and generate the human readable report according to the first image, each of the at least one second image, each quantified parameter information, each information reference value, and each information abnormality marker.

In one of the embodiments, the report generating device is further configured to present a display window displaying the second image in response to a triggering operation for the state information of the target part in the human readable report, and a target second image corresponding to the state information of the target part is shown in the display window displaying the second image.

In one of the embodiments, the report generating device is further configured to determine a third ROI in the target second image in response to a region selection operation for the target second image in a display window displaying the second image, determine a fourth ROI relevant to the third ROI in each of the at least one second image other than in the target second image, and update the human readable report according to quantified parameter information corresponding to the third ROI and quantified parameter information corresponding to the fourth ROI.

In one of the embodiments, the report generating device is further configured to present an enlarged second image in the human readable report in response to a first triggering operation for the second image in the human readable report, and rescale the enlarged second image in response to a second triggering operation for the enlarged second image.

In one of the embodiments, the report generating device is further configured to delete quantified parameter information corresponding to a target second ROI in the human readable report in response to a region deletion operation for the target second ROI in the second image in the human readable report.

In one of the embodiments, the report generating device is further configured to determine a region identifying model corresponding to the target part, and input the first image into the region identifying model corresponding to the target part to obtain the first ROI in the first image.

In a second aspect, a medical image processing method applied in the medical image processing system above, including following steps.

The first image and the second image of the target part acquired by the medical imaging device are acquired, the first image is used for depicting the anatomy structure of the target part, and the second image is used for depicting the quantified parameter information of the target part.

The first ROI in the first image is identified.

The registration is performed for the first image and the second image to obtain the second ROI in the second image, the second ROI in the second image being relevant to the first ROI in the first image.

The human readable report of the target part is generated according to the quantified parameter information corresponding to the second ROI in the second image.

In one embodiment, the performing the registration for the first image and the second image to obtain the second ROI in the second image includes: performing the registration for the first image and the second image to obtain a mapping relationship between the first image and the second image, and determining, according to the mapping relationship, an image region in the second image relevant to the first ROI to be the second ROI.

In one of the embodiments, the determining, according to the mapping relationship, an image region in the second image relevant to the first ROI to be the second ROI includes: determining first location information of the first ROI in the first image, determining, according to the mapping relationship, the second location information in the second image relevant to the first location information, and determining the second ROI in the second image according to the second location information.

In one of the embodiments, at least one second image is provided, and at least one second ROI in each of the at least one second image is provided. Each of the at least one second image is used for depicting quantified parameter information of the target part in different dimensions. The method further includes generating the human readable report of the target part based on the first image, each of the at least one second image, and each quantified parameter information corresponding to each of the at least one second ROI.

In one of the embodiments, the method further includes acquiring an information reference value corresponding to each quantified parameter information, generating an information abnormality marker in a case that the quantified parameter information does not conform to the information reference value, and generating the human readable report according to the first image, each of the at least one second image, each quantified parameter information, each information reference value, and each information abnormality marker.

In one of the embodiments, the method further includes presenting a display window displaying the second image in response to a triggering operation for the state information of the target part in the human readable report. A target second image corresponding to the state information of the target part is shown in the display window displaying the second image.

In one of the embodiments, the method further includes determining a third ROI in the target second image in response to a region selection operation for the target second image in a display window displaying the second image, determining a fourth ROI relevant to the third ROI in each of the at least one second image other than in the target second image, and updating the human readable report according to quantified parameter information corresponding to the third ROI and quantified parameter information corresponding to the fourth ROI.

In a third aspect, the present disclosure also provides a computer apparatus including a memory and a processor. A computer program is stored in the memory, and the processor, when executing the computer program, performs following steps.

The first image and the second image of the target part acquired by the medical imaging device are acquired, the first image is used for depicting the anatomy structure of the target part, and the second image is used for depicting the quantified parameter information of the target part.

The first ROI in the first image is identified.

The registration is performed for the first image and the second image to obtain the second ROI in the second image, the second ROI in the second image is relevant to the first ROI in the first image.

The human readable report of the target part is generated according to the quantified parameter information corresponding to the second ROI in the second image.

In a fourth aspect, the present disclosure further provides a non-transitory computer readable storage medium, having a computer program stored thereon. The computer program, when executed by a processor, causes the processor to perform following steps.

The first image and the second image of the target part acquired by the medical imaging device are acquired, the first image is used for depicting the anatomy structure of the target part, and the second image is used for depicting the quantified parameter information of the target part.

The first ROI in the first image is identified.

The registration is performed for the first image and the second image to obtain the second ROI in the second image, and the second ROI in the second image is relevant to the first ROI in the first image.

The human readable report of the target part is generated according to the quantified parameter information corresponding to the second ROI in the second image.

In a fifth aspect, the present disclosure also provides a computer program product, including a computer program. The computer program, when executed by a processor, causes the processor to perform following steps.

The first image and the second image of the target part acquired by the medical imaging device are acquired, the first image is used for depicting the anatomy structure of the target part, and the second image is used for depicting the quantified parameter information of the target part.

The first ROI in the first image is identified.

The registration is performed for the first image and the second image to obtain the second ROI in the second image, and the second ROI in the second image is relevant to the first ROI in the first image.

The human readable report of the target part is generated according to the quantified parameter information corresponding to the second ROI in the second image.

In the medical image processing system, the method, the computer apparatus, the storage medium, and the computer program product are described above, the medical imaging device acquires the first image and the second image of the target part through the medical imaging device, and transmits the first image and the second image to the report generating device, and the report generating device identifies the first ROI in the first image, performs the registration on the second image to the first image, and determines the second ROI in the second image, and generates the human readable report of the target part according to the quantified parameter information corresponding to the second ROI in the second image. In the case that the first ROI may be easily identified from the first image, the ROI may be synchronously updated in the second image through the registration for the images, thereby reducing complexity of acquiring the second ROI in the second image, and making a medical image processing performed for the second ROI in the second image more convenient.

### BRIEF DESCRIPTION OF THE DRAWINGS

FIG. 1 is a block view illustrating a structure of a medical image processing system according to an embodiment.

FIG. 2 is a schematic view illustrating an application environment of a medical image process according to an embodiment.

FIG. 3 is a schematic flow chart of generating a human readable liver comprehensive report according to an embodiment.

FIG. 4 is a schematic view of a qualitative image and quantitative images according to an embodiment.

FIG. 5 is a schematic view illustrating the human readable liver comprehensive report according to an embodiment.

FIG. 6 is a schematic view showing an enlarged quantitative image according to an embodiment.

FIG. 7 is a schematic view showing synchronously updated regions of interest (ROIs) according to an embodiment.

FIG. 8 is a schematic flow chart of a medical image processing method according to an embodiment.

FIG. 9 is a view showing an internal structural of a computer apparatus according to an embodiment.

### DETAILED DESCRIPTION OF THE EMBODIMENTS

In order to make the objectives, the technical solutions, and the advantages of the present disclosure clearer and to be better understood, the present disclosure will be further described in detail with reference to the accompanying drawings and the embodiments. It should be understood that the specific embodiments described herein are merely used to illustrate the present disclosure but not intended to limit the present disclosure.

Currently, for lesion areas in a qualitative image and a quantitative image, an ROI is manually circled for each image, and after the ROI is processed, the human readable report corresponding to each image is generated. The generation method of the human readable report is cumbersome to perform, and the diagnostic information is scattered, thus making it inconvenient to diagnose the disease by using multi-dimensional diagnostic information. Therefore, the conventional medical image processing technology has a problem that it is cumbersome to perform.

In an embodiment of the present disclosure, as shown in FIG. 1, a medical image processing system is provided. The system includes a medical imaging device 102 and a report generating device 104. The medical imaging device 102 may be, but is not limited to, a Magnetic Resonance Imaging (MM) device, a Positron Emission Computed Tomography (PET) devices, and a combined device (PET-MRI) of PET and MRI. The report generating device 104 may be, but is not limited to, a personal computer, a notebook computer, a smartphone, a tablet computer, an Internet of Things (IoT) device, and a portable wearable devices. The IoT device may be a smart speaker, a smart television, a smart air conditioner, a smart vehicle-mounted device, and the like. The portable wearable device may be a smart watch, a smart bracelet, a head-wearing device, and the like.

The medical imaging device 102 is configured to acquire a first image and a second image of a target part, and transmit the first image and the second image to the report generating device 104. The first image is used for depicting an anatomy structure of the target part, and the second image is used for depicting quantified parameter information of the target part.

The report generating device 104 is configured to identify a first ROI in the first image, and perform a registration for the first image and the second image to obtain a second ROI in the second image. The second ROI in the second image is relevant to the first ROI in the first image.

The report generating device 104 is further configured to generate human readable report of the target part according to quantified parameter information corresponding to the second ROI in the second image.

The target part may be a body part to be diagnosed.

The first image may be a qualitative image, i.e. a structure image, used for describing an anatomical structure of the body part to be diagnosed. The first image may be a T1-weighted image, a T2-weighted image, or a diffusion weighted imaging (DWI) image, acquired by an MRI device.

The second image may be a quantitative image used for describing physiological conditions of the body part to be diagnosed. The second image may be a fat analysis and calculation technology (FACT) image, a susceptibility weighted imaging (SWI), a spin-lattice relaxation time (T1ρ) image, a relaxation time mapping image (T1/T2/T2* Mapping image), a magnetic resonance elastography (MRE) image, or a fluid attenuated inversion recovery (FLAIR) image, acquired by the MRI device, and the second image may also be a PET image acquired by the PET or PET-MRI device.

An R2* parameter diagram is generated simultaneously during acquisition of the FACT image. Illustratively, when the FACT quantitative image is scanned, a multi-parameter water map, a fat map, an in-phase (IP) image, an out-of-phase (OP) image, a fat fraction (FF) image, and the like, are outputted.

The quantization parameters, the structure parameters, or the contrast ratios of the same tissue of the target part that can be presented by the first image and the second image are different. Taking a magnetic resonance scan as an example, the first image and the second image may be obtained by exciting the target part through using different imaging sequences, respectively. For example, the target part is the cerebrospinal fluid in the brain, the first image uses a T1WI (T1 weighted imaging) sequence, and the second image uses a T2WI (T2 weighted imaging) sequence. The corresponding cerebrospinal fluid region is characterized as high signals in the second image and low signals in the first image. For another example, the first image is a diffusion weighted imaging (DWI) image obtained by using a DWI sequence, and the second image is an apparent diffusion coefficient (ADC) image created by mathematically removing a T2 effect from the DWI image. Whether a diffusion is limited or not can be determined by the DWI image and the ADC image, moreover, a T2 penetration effect, a T2 clearance effect, and a T2 darkening effect, and the like occurring in the DWI image can be determined in combining with the ADC image.

The first ROI and the second ROI may be lesion regions of the body part to be diagnosed.

The quantified parameter information may be a measure value of the physiological index of the lesion region, for example, a degree of iron deposition or a fat content in the liver lesion region, or average and/or maximum standardized uptake values (SUV).

The human readable report may be a readable text report, a text report with a digital image attached thereto, or a text report with information such as a subsequent medical treatment or examination suggestion.

In a specific implementation, the medical imaging device may acquire the qualitative images and the quantitative images of the body part to be diagnosed and send the acquired qualitative images and quantitative images to a report generating device. After receiving the qualitative images and the quantitative images, the report generating device may intelligently identify the lesion region from the qualitative images and use the identified lesion region as the first ROI. The report generating device may also perform a registration for the qualitative image and the quantitative image, so that there is a certain mapping relationship between pixel coordinates of the qualitative image and pixel coordinates of the quantitative image, and the second ROI in the quantitative image corresponding to the first ROI may be determined according to the mapping relationship. The second ROI and the first ROI may correspond to the same lesion region. The report generating device may also acquire the measured value of the human physiological index for the second ROI in the quantitative image, and generate a human readable comprehensive report of the body part to be diagnosed according to the measured value.

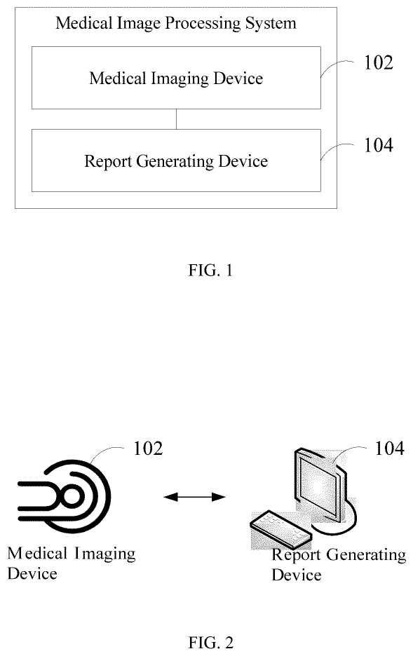

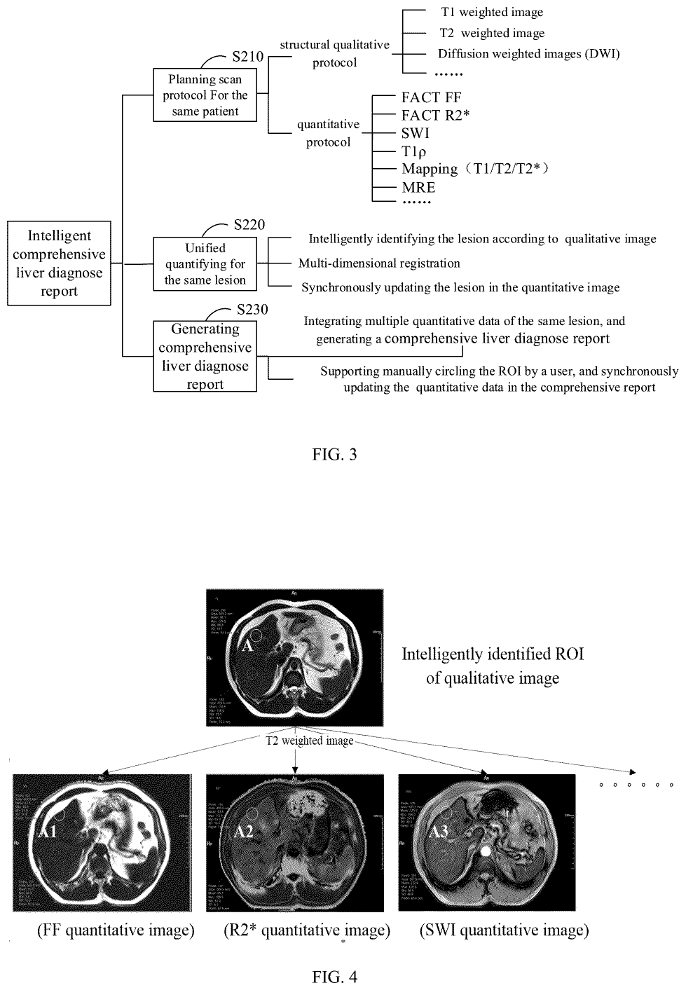

FIG. 2 is a schematic view illustrating a disclosure environment of a medical image process. The medical imaging device 102 communicates with the report generating device 104 via a wired or wireless link. FIG. 3 is a schematic flow chart of generating human readable liver comprehensive report. According to FIG. 3, the generation of the human readable liver comprehensive report may include the following steps S210 to S230.

At step S210, a scan protocol is planned for a patient for conditions of the patient. The scan protocol may include a structural qualitative protocol and a quantitative protocol. The structural qualitative protocol may include scan protocols of images, such as a T1 contrast ratio image, a T2 contrast ratio image, and a DWI image, etc. The quantitative protocol may include scan protocols of images, such as a FACT FF (FF for short) image, a FACT R2* (R2* for short) image, a SWI image, a T1ρ image, a Mapping (T1/T2/T2*) image, a MRE image, and a FLAIR image, etc.

At step S220, the ROI identified from the qualitative image is updated synchronously to a multi-dimensional quantitative image. Specifically, according to the planned scan protocol, a qualitative image and a plurality of quantitative images of a patient's liver may be obtained. The lesion region may be intelligently identified from the qualitative image, and the identified lesion region is used as the ROI. A registration is performed for the qualitative image and the plurality of quantitative images, so that there is a certain mapping relationship between the pixel coordinates of the qualitative image and the pixel coordinates of each quantitative image. The ROI in the qualitative image is synchronously mapped to each quantitative image according to the mapping relationship, thereby obtaining the ROI in each quantitative image.

At step S230, a human readable liver comprehensive report is generated according to the ROI in each quantitative image. Specifically, the ROI in each quantitative image may correspond to the same lesion region, and the measured value of the ROI in each quantitative image is acquired. Different measured values may reflect different physiological indexes of the same lesion region. The human readable comprehensive report of the liver may be generated by integrating the measured values into the same human readable report. The human readable comprehensive report may present the qualitative image and the quantitative image. If the user is not satisfied with the result currently presented by the human readable report, a new ROI may also be selected by manually circling from the qualitative image or the quantitative image presented in the human readable report, and the measured value corresponding to the new ROI may be synchronously updated in the human readable report.

FIG. 4 is a schematic view showing a qualitative image and quantitative images. According to FIG. 4, the qualitative image may be the T2 contrast ratio image, and the quantitative images may include the FF image, the R2* image, and the SWI image, etc. The lesion region A may be intelligently identified from the T2 contrast ratio image to act as the ROI, and the multi-dimensional registration is performed for the qualitative image and the quantitative images, and the ROI is synchronously applied to the quantitative images to obtain the ROIs A1, A2, and A3 in the FF image, in the R2* image, and in the SWI image, respectively. Where A, A1, A2, and A3 may correspond to the same lesion region of interest, each qualitative image or quantitative image may contain multiple ROIs.

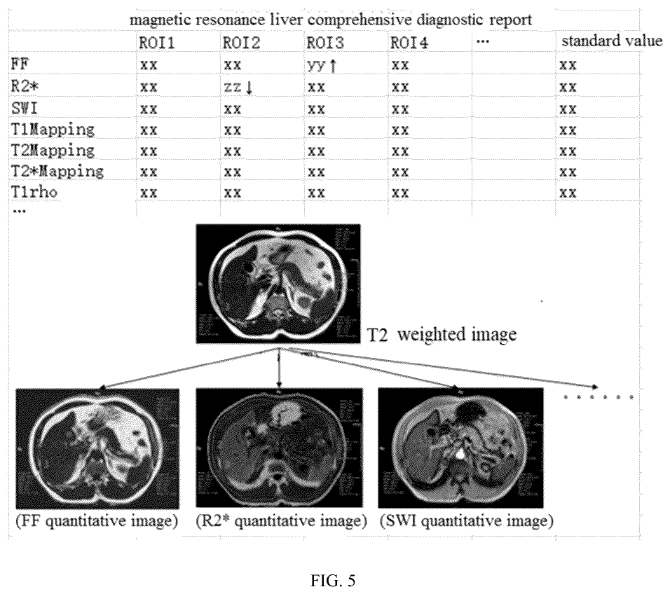

FIG. 5 is a schematic view showing the human readable liver comprehensive report. According to FIG. 5, a measured value of each ROI in each quantitative image may be obtained, and the measured values are integrated in the same human readable report to generate the human readable liver comprehensive report. The qualitative image, the quantitative images, a measured value of each ROI in each quantitative image, and a standard value corresponding to each measured value may be presented in the human readable comprehensive report. The human readable comprehensive report may also present a comparison result of the measured value and the standard value. In the case that the measured value does not match with the standard value, the measured value may be presented in red. If the measured value is greater than the standard value, a marker denoting larger value may be added to the measured value. If the measured value is less than the standard value, a marker denoting smaller value may be added to the measured value. For example, a sign ↑ in FIG. 5 indicates that the measured value is greater than the standard value, and a sign ↓ indicates that the measured value is less than the standard value. In the human readable comprehensive report, the standard value and the plurality of measured values corresponding to the same quantitative image may be in the same row, the plurality of measured values corresponding to the same lesion region of interest may be in the same column. For example, the measured values of the regions of interest ROI1, ROI2, ROI3, and ROI4 in the FF image and the standard value of the FF image may be in the same row, and the measured values of the region of interest ROI1 in the FF image, the R2* image, the SWI image, the T1/T2/T2* Mapping image, and the T1ρ image may be in the same column.

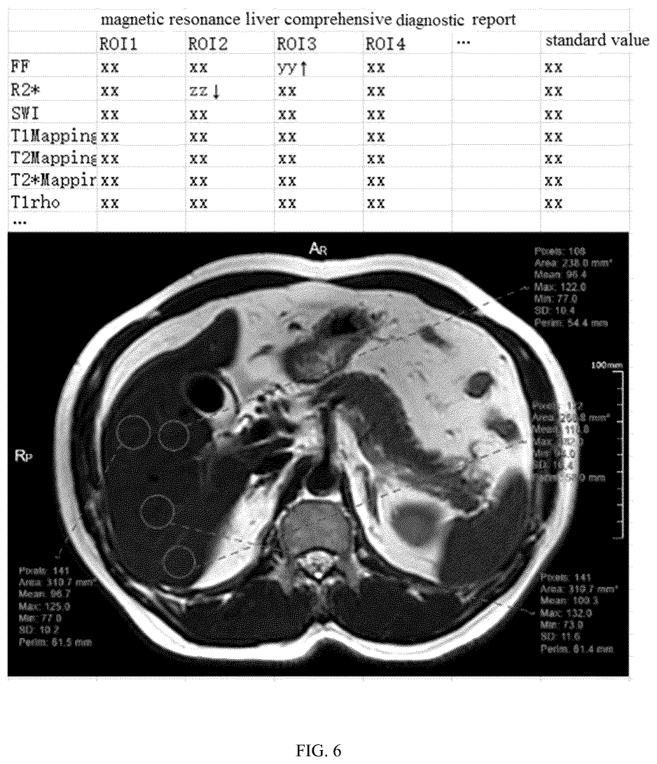

FIG. 6 is a schematic view showing an enlarged quantitative image. According to FIG. 6, one qualitative image or quantitative image in the human readable comprehensive report may be triggered to be enlarged, and may be restored when triggered again. For example, the FF image in the human readable comprehensive report is double-clicked to obtain an enlarged FF image. As shown in FIG. 6, by double-clicking the enlarged FF image again, the FF image may be reduced to an original size and the human readable comprehensive report is restored.

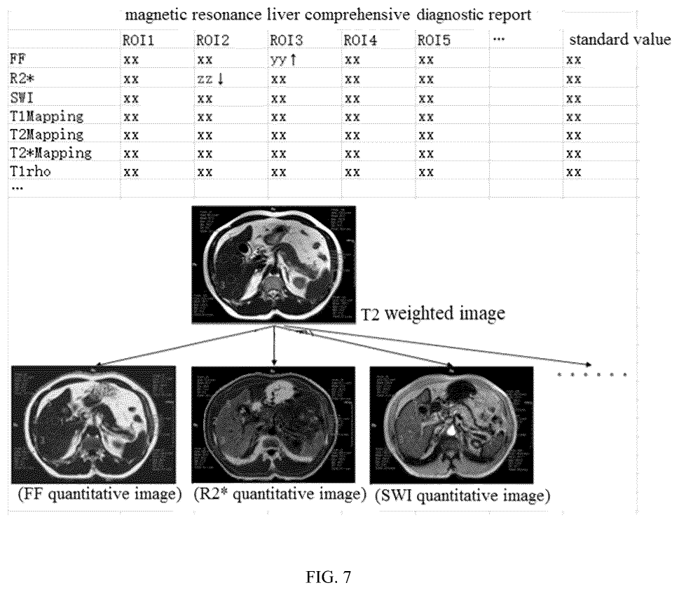

FIG. 7 is a schematic view showing synchronously updated ROIs. According to FIG. 7, any measured value in the human readable comprehensive report may be triggered to open the quantitative image corresponding to the measured value, and by circling a new ROI in the quantitative image, the new ROI may be updated synchronously in other quantitative images, the measured values corresponding to the new ROIs in all quantitative images may be acquired, and the measured values corresponding to the new ROIs are added to the human readable comprehensive report. For example, measured values of four regions of interest ROI1, ROI2, ROI3, and ROI4 are given in the current human readable report. If a doctor is not satisfied with the result presented in the current human readable report, any measured value corresponding to the FF image may be triggered to open the FF image. The doctor may manually circle a new region of interest ROI5 in the opened FF image, and the ROI5 may be synchronously updated in the R2* image, the SWI image, the T1/T2/T2* Mapping image, and the T1ρ image. The measured values of the ROI5 in the FF image, the R2* image, the SWI image, the T1/T2/T2* Mapping image, and the T1ρ image are acquired to form a new column of measured results of the ROI5 and added to the human readable comprehensive report.

The medical image processing system acquires the first image and the second image of the target part through the medical imaging device, and transmits the first image and the second image to the report generating device. The report generating device identifies the first ROI in the first image, performs the registration on the second image to the first image, and determines the second ROI in the second image, and generates the human readable report of the target part according to the quantified parameter information corresponding to the second ROI in the second image. In the case that the first ROI may be easily identified from the first image, the ROI may be synchronously updated in the second image through the registration for the images, thereby reducing complexity of acquiring the second ROI in the second image, and making a medical image processing performed for the second ROI in the second image more convenient.

In an embodiment, the report generating device above is further configured to perform the registration for the first image and the second image, to obtain a mapping relationship between the first image and the second image. According to the mapping relationship, an image region in the second image relevant to the first ROI is determined to be the second ROI.

In a specific implementation, the report generating device may select marker points for the same anatomical position of the human body in the first image and in the second image, respectively, to obtain a first marker point in the first image and a second marker point in the second image, and take a mapping relationship between the spatial coordinates of the first marker point and the spatial coordinates of the second marker point as the mapping relationship between the first image and the second image. After the first ROI in the first image is determined, the coordinates of points corresponding to the first ROI may be obtained, and coordinates of points of the second ROI corresponding to the coordinates of points of the first ROI are determined in the second image according to the mapping relationship, and a region including the coordinates of points of the second ROI in the second image is used as the second ROI.

For example, for the same anatomical position of the human body, a marker point x in the qualitative image is selected, a marker point y in the quantitative image is selected, and the mapping relationship y=f (x) between the marker points x and y is used as the mapping relationship between the qualitative image and the quantitative image. After the first ROI in the qualitative image is determined, the coordinates of points x₁, x₂, . . . , x_{N} on the boundary of the first ROI may be obtained and are substituted into the mapping relationship y=f (x) to obtain points y₁, y₂, . . . , y_{N}, and the points y₁, y₂, . . . , y_{N} in the quantitative image are connected to obtain the boundary of the second ROI, and the second ROI may be the boundary and an inside of the boundary.

In the present embodiment, the mapping relationship between the first image and the second image is obtained by performing the registration for the first image and the second image, and the image region in the second image, which is relevant to the first ROI, is determined to be the second ROI according to the mapping relationship. On the basis of performing the registration for the first image and the second image, the ROI in the first image may be synchronously updated in the second image, thereby increasing the convenience of obtaining the second ROI.

In an embodiment, the report generating device above is further configured to determine a first location information of the first ROI in the first image. A second location information relevant to the first location information is determined in the second image according to the mapping relationship. The second ROI in the second image is determined according to the second location information.

In a specific implementation, the report generating device may select the point to be matched in the first ROI of the first image, and the position coordinates of the point to be matched are used as the first location information. The second location information corresponding to the first location information is determined according to the mapping relationship, and the target point in the second image is determined according to the second location information, and the region corresponding to the target point is used as the second ROI.

For example, the boundary points of the first ROI may be selected to act as the points to be matched to obtain the first location information x₁, x₂, . . . , x_{N}, and the first location information is substituted into the mapping relationship y=f(x) to obtain the second location information y₁, y₂, . . . , y_{N}. The points corresponding to y₁, y₂, y_{N} are the target points, and the target points are connected in the second image to obtain the boundary of the second ROI, and the second ROI may be the boundary and the inside of the boundary.

In this embodiment, by determining the first location information of the first ROI in the first image, the second location information relevant to the first location information is determined in the second image according to the mapping relationship. The second ROI in the second image is determined according to the second location information. For the same lesion, the ROIs in the first image and in the second image may be determined, respectively, thereby obtaining the multi-dimensional information of the same lesion, and identifying condition of the lesion accurately.

In an embodiment, at least one second image is provided, and at least one second ROI in each second image is provided. Each second image is used for depicting quantified parameter information of the target part in different dimensions. The report generating device is further configured to generate the human readable report of the target part based on the first image, each second image, and the quantified parameter information corresponding to each second ROI.

In a specific implementation, one or more quantitative images may be acquired, each quantitative image may include one or more ROIs, and each quantitative image may correspond to a different human physiological index. The report generating device may acquire the measured value of the human physiological index of each ROI in each quantitative image, and present the qualitative image, one or more quantitative images, and the measured value of each ROI in each quantitative image in the generated human readable report.

In this embodiment, the human readable report of the target part is generated according to the quantified parameter information corresponding to the first image, each second image, and each second ROI, and the human physiological indexes may be presented in multiple dimensions in the generated human readable report, so that the lesion condition may be accurately identified.

In an embodiment, the report generating device is further configured to acquire an information reference value corresponding to the quantified parameter information. In the case that quantified parameter information does not conform to the information reference value, an information abnormality marker is generated. The human readable report is generated according to the first image, each second image, each quantified parameter information, each information reference value, and each information abnormality marker.

In a specific implementation, a standard value corresponding to a measured value of the human physiological index may be stored in the report generating device in advance. If identifying that the measured value does not match the standard value, the report generating device may generate the information abnormality marker, and present the qualitative image, one or more quantitative images, the measured value of each ROI in each quantitative image, the standard value corresponding to each ROI in each quantitative image, and the information abnormality marker in the generated human readable report.

For example, the standard value may be a value or a value interval. If the measured value is not equal to the standard value, or if the measured value does not fall within the standard value interval, the measured value may be marked in red. If the measured value is greater than the standard value, a marker denoting a larger value may be added to the measured value. If the measured value is less than the standard value, a marker denoting less value may be added to the measured value. Finally, the qualitative image, the quantitative images, the measured value, the standard value, the red measured value, the marker denoting greater value, and the marker denoting less value may be presented in the human readable comprehensive report.

In this embodiment, the information reference value corresponding to the quantified parameter information is acquired, and in the case that the quantified parameter information does not conform to the information reference value, the information abnormality identification is generated, and the human readable report is generated according to the first image, each second image, each quantified parameter information, each information reference value, and the information abnormality identification. The human readable report may give a reminder when the quantified parameter information does not conform to the reference value, so that the abnormality of the human body part may be found in time, thereby improving the recognition efficiency for the lesion condition.

In an embodiment, the report generating device above is further configured to present a display window displaying the second image in response to a triggering operation for the state information of the target part in the human readable report. The target second image corresponding to the state information of the target part is shown in the display window displaying the second image.

In a specific implementation, the user may select the target measured value from the human readable report, and the report generating device generates a new image showing window for presenting the quantitative image corresponding to the target measured value in response to the triggering operation of the user for the target measured value.

For example, according to FIG. 7, if the user is not satisfied with the result currently presented by the human readable report, the user may double-click any measured value corresponding to the FF image, and the report generating device may generate a new window to present the FF image in response to the double click operation.

In this embodiment, the display window displaying the second image is showed in response to the triggering operation for the state information of the target part in the human readable report, so that the user may conveniently open the second image corresponding to the state information of the target part and view the second image, thereby increasing the convenience of use for the user.

In an embodiment, the report generating device above is further configured to determine a third ROI in the target second image in response to a region selection operation for the target second image in the display window displaying the second image, and a fourth ROI relevant to the third ROI is determined in each second image other than in the target second image. The human readable report is updated according to the quantified parameter information corresponding to the third ROI and the quantified parameter information corresponding to the fourth ROI.

In a specific implementation, after the report generating device generates the new image showing window in response to a user's triggering operation for the target measured value to present the quantitative image corresponding to the target measured value, the user may select a new ROI in the quantitative image presented in the new image showing window, and the report generating device generates the third ROI in the quantitative image presented in the new image showing window in response to the user's selection operation for a new ROI, and the third ROI is synchronously updated in other quantitative images to form a fourth ROI. The report generating device may also acquire measured values of the third ROI and the fourth ROI, and updates each measured value in the human readable report.

For example, according to FIG. 7, after the report generating device generates the new window in response to the user's double click operation to present the FF image, the user may also select a new region of interest ROI5 in the presented FF image by dragging a mouse, and the report generating device may synchronously update the new region of interest ROI5 in the R2* image, in the SWI image, in the T1/T2/T2* Mapping image, and in the T1ρ image, and acquire the measured values corresponding to the ROI5 in each of the FF image, the R2* image, the SWI image, the T1/T2/T2* Mapping image, and the T1ρ image, and may update the measured values of the ROI5 in the human readable report by adding a column in the human readable report.

In this embodiment, the third ROI in the target second image is determined in response to the region selection operation for the target second image in the display window displaying the second image, the fourth ROI relevant to the third ROI is determined in each second image other than in the target second image. The human readable report is updated according to the quantified parameter information corresponding to third ROI and the quantified parameter information corresponding to the fourth ROI, which enables the measured values of other ROIs to be synchronously updated in the human readable report when the user needs to view the results of the other ROIs, thereby increasing operation convenience.

In an embodiment, the above report generating device is further configured to present an enlarged second image in the human readable report in response to a first triggering operation for the second image in the human readable report, and reduce the enlarged second image in response to a second triggering operation for the enlarged second image.

In a specific implementation, the user may trigger the quantitative images in the human readable report, and the report generating device may present the enlarged quantitative image in the human readable report in response to the user's triggering operation. The user may also trigger the enlarged quantitative image, and the report generating device may also reduce the enlarged quantitative image in response to the user's triggering operation.

For example, according to FIG. 6, the user may double-click the FF image in the human readable report to generate an enlarged FF image in the human readable report, and the user may also double-click the enlarged FF image, so that the enlarged FF image is reduced to an original size, and that the human readable report is restored.

In the present embodiment, the enlarged second image is presented in the human readable report in response to the first triggering operation for the second image in the human readable report, and the enlarged second image is reduced in response to the second triggering operation for the enlarged second image, so that the quantitative image presented in the human readable report may be enlarged and restored, thereby making it easy for the user to view the second image.

In an embodiment, the report generating device above is further configured to delete the quantified parameter information corresponding to the target second ROI in the human readable report in response to a region deletion operation for the target second ROI in the second image in the human readable report.

In a specific implementation, the user may perform a region deletion operation on any ROI in the quantitative images in the human readable report, and the report generating device may delete a measured value corresponding to the ROI in the human readable report in response to the user's region deletion operation.

For example, according to FIG. 5, a region deletion operation may be formed by the user single-clicking and selecting the region of interest ROI1 in the FF image and then pressing the Delete button on the keyboard, and the report generating device may delete the column corresponding to the ROI1 in the measured values in the human readable report in response to the user's region deletion operation.

In this embodiment, the quantified parameter information corresponding to the target second ROI in the human readable report is deleted in response to the region deletion operation for the target second ROI in the second image in the human readable report, so that the user may flexibly configure items in the human readable report, thereby increasing flexibility in generating the human readable report.

In an embodiment, the report generating device is further configured to determine a region identifying model corresponding to the target part, input the first image into the region identifying model corresponding to the target part to obtain the first ROI in the first image.

The region identifying model may be a machine learning model.

In a specific implementation, the report generating device may determine a machine learning model adaptive for the human body part to be diagnosed, and input the quantitative image into the machine learning model, and identify a lesion region of the ROI in the quantitative image.

In the present embodiment, by determining the region identifying model corresponding to the target part, and inputting the first image into the region identifying model corresponding to the target part, the first ROI in the first image is obtained. Since the first image facilitates description of the anatomical structure of the body part to be diagnosed, the lesion region of the part to be diagnosed may be quickly and accurately identified, thereby ensuring the efficiency and accuracy of the determination of the ROI.

In an embodiment, the first image includes at least one of a T1 contrast ratio image, a T2 contrast ratio image, and a DWI image. The second image includes at least one of an FACT image, an SWI image, a T1ρ image, a T1/T2/T2* Mapping image, an MRE image, and a PET image.

In a specific implementation, the first image may be the T1 contrast ratio image, the T2 contrast ratio image, or the DWI image acquired by the MRI device. The second image may be the FACT FF image, the FACT R2* image, the SWI image, the T1ρ image, the T1/T2/T2* Mapping image, the MRE image, or the FLAIR image acquired by the MRI device, and the second image may also be the PET image acquired by the PET device or by the PET-MR device.

In the present embodiment, by setting the first image and the second image, the ROI in the second image may be quickly and accurately determined based on the first image accurately describing the anatomical structure of the human body, thereby ensuring the efficiency and accuracy of the determination of the ROI.

In order to make the embodiments of the present disclosure to be thoroughly understood by those skilled in the art, the present disclosure will be illustrated in conjunction with a specific example.

With the development of magnetic resonance sequence technology, the magnetic resonance scanning technology for liver can not only be used for a multi-contrast qualitative diagnosis, but also for a multi-dimensional quantitative diagnosis. Therefore, it has become an urgent technique required to qualitatively locate the lesions and synthesize quantitative data to form a human readable comprehensive report. In the prior art, the ROI is manually circled and then the regions are post-processed one after another to form a plurality of human readable report. Such an operation is not only cumbersome, but also not easy for a subsequent viewing. At the same time, the diagnostic information is scattered, which is unfavorable for the user to synthesize the information of all parties to diagnose the disease. In view of this, a quick method for intelligently identifying a lesion while ensuring that multi-dimensional data all come from the same ROI and that a registration is performed for all dimensions to form an online multi-dimensional comparative quantitative comprehensive report. This method can not only reduce the manual operation to save time, but also make it easy for the doctor to view the human readable report, thereby improving the work efficiency.

Specifically, the lesion ROI may be intelligently identified from the qualitative image, and the ROI is synchronously applied to the quantitative image through the multi-dimensional registration, and a uniform human readable comprehensive report is generated according to the measured values in the quantitative image. As shown in FIG. 5, the human readable report may reflect different quantitative results corresponding to the ROIs, and a specification of standard value is attached thereto. If the quantitative results are not within the standard value range, the quantitative results may be presented in red and are marked with a downward or upward arrow.

The qualitative image or quantitative images may be shown in the human readable report as reference images. Double-clicking any reference image may enlarge the reference image. As shown in FIG. 6, double-clicking the image again may make the enlarged reference image restore.

The user may also manually double-click any value in the human readable report to open the image corresponding to the value. The user may manually circle an ROI in the image, and the ROI may be synchronously updated to all quantitative images, and the data in the human readable report are synchronously updated, and the final report may be as shown in FIG. 7.

It should be noted that, for the PET-MR, based on the technical solution in the above-described embodiments of the present disclosure, an SUV value of an ROI in the PET image may also be synchronously updated in the human readable comprehensive report by using the MR image as a qualitative image and using the PET image as a quantitative image, and by performing a registration for the PET image and the MR image.

In the present embodiment, the ROI is intelligently identified, which enables the multi-dimensional quantitative data to use a common and uniform ROI. By integrating the multi-dimensional quantitative data, a liver comprehensive text report quantitative data may be generated. Furthermore, the user may adjust, add or delete an ROI, and dynamically update the comprehensive report data, thereby improving flexibility of generating a report.

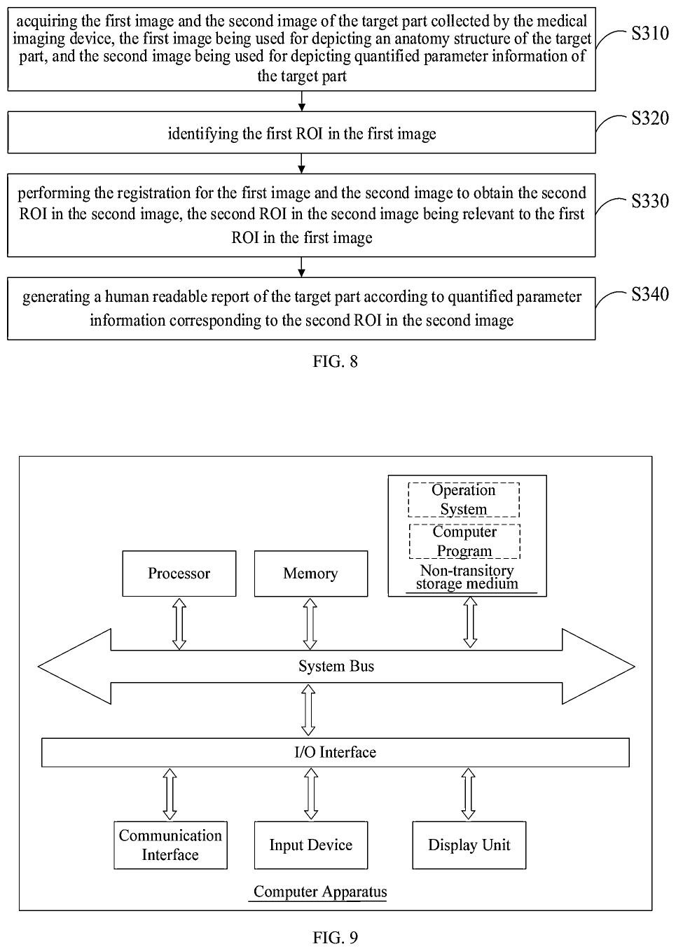

In an embodiment, as shown in FIG. 8, a medical image processing method is provided. Taking the method applied in the medical image processing system in FIG. 1 as an example, the executing subject of the method may be the report generating device 104 of the medical image processing system, and the method includes following steps.

In step S310, a first image and a second image of a target part acquired by a medical imaging device 102 are acquired. The first image is used for depicting an anatomy structure of a target part, and the second image is used for depicting quantified parameter information of the target part.

In step S320, a first ROI in the first image is identified.

In step S330, a registration is performed for the first image and the second image to obtain a second ROI in the second image. The second ROI in the second image is relevant to the first ROI in the first image.

In step S340, human readable report of the target part is generated according to the quantified parameter information corresponding to the second ROI in the second image. diagnostic

In a specific implementation, the medical imaging device may acquire the qualitative images and the quantitative images of the body part to be diagnosed and send the acquired qualitative images and the quantitative images to a report generating device. After receiving the qualitative images and the quantitative images, the report generating device may intelligently identify the lesion region from the qualitative images and use the identified lesion region as the first ROI. The report generating device may also perform a registration for the qualitative image and the quantitative image, so that there is a certain mapping relationship between pixel coordinates of the qualitative image and pixel coordinates of the quantitative image, and the second ROI in the quantitative image corresponding to the first ROI may be determined according to the mapping relationship. The second ROI and the first ROI may correspond to the same lesion region. The report generating device may also acquire the measured value of the human physiological index for the second ROI in the quantitative image, and generate a human readable comprehensive report of the body part to be diagnosed according to the measured value.

Since the processing procedure of the report generating device 104 has been described in detail in the embodiment above, it will not be described repeatedly hereinafter.

In this embodiment, the first image and the second image of the target part acquired by the medical imaging device are acquired. The first ROI in the first image is identified, the registration is performed for the second image and the first image, and the second ROI in the second image is determined. The human readable report of the target part is generated according to the quantified parameter information corresponding to the second ROI in the second image. In the case that the first ROI is easily identified from the first image, the ROI may be updated in the second image synchronously through the registration for the images, thereby reducing complexity of acquiring the second ROI in the second image, and making it more convenient to perform a medical image processing for the second ROI in the second image.

It should be understood that although the steps in the flow charts of all embodiments are shown sequentially as indicated by arrows, these steps are not necessarily performed sequentially as indicated by arrows. Unless expressly stated herein, these steps are not performed in a strict order, and may be performed in other orders. Moreover, at least a portion of the steps included in all embodiments may include a plurality of sub-steps or a plurality of stages, which are not necessarily performed at the same time, but may be performed at different time, and the sub-steps or stages may not necessarily be performed sequentially, but may be performed sequentially or alternately with other steps, or with at least a portion of the sub-steps or stages of other steps.

In one of the embodiments, a computer apparatus is provided. The computer apparatus may be a terminal, the internal structure of which is shown in FIG. 9. The computer apparatus includes a processor, a memory, a communication interface, a display screen, and an input device which are connected by a system bus. The processor of the computer apparatus is configured to provide computing and control capabilities. The memory of the computer apparatus includes a non-transitory storage medium and an internal memory. The non-transitory storage medium stores an operating system and a computer program. The internal memory provides an environment for the operation of an operating system and a computer program in a non-transitory storage medium. The communication interface of the computer apparatus is used for wire or wireless communication with external terminals, and the wireless communication may be implemented by WIFI, mobile cellular network, NFC (near field communication) or other technologies. The computer program, when executed by the processor, performs the medical image processing method. The display screen of the computer apparatus may be a liquid crystal display screen or an electronic ink display screen, and the input device of the computer apparatus may be a touch layer covered on the display screen, or may be a key, a trackball or a touch pad provided on the housing of the computer apparatus, or may be an external keyboard, touch pad or mouse.

It should be understood by those skilled in the art that the structure shown in FIG. 9 is a block diagram showing only part of the structure relevant to the solutions of the present disclosure, but not intended to limit the computer apparatus to which the solutions of the present disclosure are applied, and that the particular computer apparatus may include more or less components than those shown in the figure, or may combine with certain components, or may have different component arrangements.

In one of the embodiments, a computer apparatus is provided. The computer apparatus includes a memory having a computer program stored therein, and a processor. The processor, when executing the computer program, performs the steps of the method embodiments described above.

In one of the embodiments, a non-transitory computer readable storage medium is provided, and a computer program is stored on the non-transitory computer readable storage medium. The computer program, when executed by a processor, performs the steps in the method embodiments above.

In one of the embodiments, a computer program product is provided and includes a computer program. The computer program, when executed by a processor, performs the steps in the method embodiments above.

A person of ordinary skill in the art may understand that all or part of the processes in the methods of the above embodiments may be achieved by the relevant hardware instructed by the computer programs. The computer programs may be stored in a non-transitory computer readable storage medium, and when being executed, perform the processes such as those of the methods of the embodiments described above. The memory, database, or other medium recited in the embodiments of the disclosure include at least one of non-transitory and transitory memory. Non-transitory memory includes read-only memory (ROM), magnetic tape, floppy disk, flash memory, optical memory, high density embedded non-transitory memory, resistive random access memory (ReRAM), magnetoresistive random access memory (MRAM), ferroelectric random access memory (FRAM), phase change memory (PCM), or graphene memory, etc. Transitory memory includes random access memory (RAM) or external cache memory, etc. For illustration rather than limitation, RAM may be in various forms, such as static random access memory (SRAM) or dynamic random access memory (DRAM), etc. The databases involved in the embodiments of the present disclosure may include at least one of a relational database and a non-relational database. The non-relational databases may include, but are not limited to, a block chain-based distributed database, etc. The processors involved in the embodiments of the present disclosure may be but are not limited to general purpose processors, central processing units, graphics processors, digital signal processors, programmable logicians, quantum computing-based data processing logicians, etc.

The technical features of the foregoing embodiments may be arbitrarily combined. For brevity, not all possible combinations of the technical features in the foregoing embodiments are described. However, the combinations of these technical features should be considered to be included within the scope of the present disclosure, as long as the combinations are not contradictory.

The above described embodiments are several implementations of the present disclosure, and the description thereof is specific and detailed, but cannot be construed as a limitation to the scope of the present disclosure. It should be noted that for a person of ordinary skill in the art, various modifications and improvements may be made without departing from the concept of the present disclosure, and all these modifications and improvements are all within the protection scope of the present disclosure. Therefore, the protection scope of the present disclosure shall be subject to the attached claims.

What is claimed is:

1. A medical image processing system, comprising a medical imaging device and a report generating device, wherein:
the medical imaging device is configured to acquire a first image and a second image of a target part, and transmit the first image and the second image to the report generating device; the first image is used for depicting an anatomy structure of the target part, and the second image is used for depicting quantified parameter information of the target part;
the report generating device is configured to identify a first region of interest (ROI) in the first image, and perform a registration for the first image and the second image to obtain a second ROI in the second image; the second ROI in the second image is relevant to the first ROI in the first image;
the report generating device is further configured to generate a human readable report of the target part according to quantified parameter information corresponding to the second ROI in the second image.

2. The system of claim 1, wherein the report generating device is further configured to:
perform the registration for the first image and the second image to obtain a mapping relationship between the first image and the second image; and
determine, according to the mapping relationship, an image region in the second image relevant to the first ROI to be the second ROI.

3. The system of claim 2, wherein the report generating device is further configured to:
determine first location information of the first ROI in the first image;
determine, according to the mapping relationship, second location information in the second image relevant to the first location information; and
determine the second ROI in the second image according to the second location information.

4. The system of claim 1, wherein:
at least one second image is provided, and at least one second ROI in each of the at least one second image is provided;
each of the at least one second image is used for depicting quantified parameter information of the target part in different dimensions;
the report generating device is further configured to generate the human readable report of the target part based on the first image, each of the at least one second image, and each quantified parameter information corresponding to each of the at least one second ROI.

5. The system of claim 4, wherein the report generating device is further configured to:
acquire an information reference value corresponding to each quantified parameter information;
generate an information abnormality marker in a case that the quantified parameter information does not conform to the information reference value; and
generate the human readable report according to the first image, each of the at least one second image, each quantified parameter information, each information reference value, and each information abnormality marker.

6. The system of claim 5, wherein the report generating device is further configured to present a display window displaying the second image in response to a triggering operation for the state information of the target part in the human readable report; and
a target second image corresponding to the state information of the target part is shown in the display window displaying the second image.

7. The system of claim 5, wherein the report generating device is further configured to:
determine a third ROI in the target second image in response to a region selection operation for the target second image in a display window displaying the second image;
determine a fourth ROI relevant to the third ROI in each of the at least one second image other than in the target second image; and
update the human readable report according to quantified parameter information corresponding to the third ROI and quantified parameter information corresponding to the fourth ROI.

8. The system of claim 5, wherein the report generating device is further configured to:
present an enlarged second image in the human readable report in response to a first triggering operation for the second image in the human readable report; or
reduce the enlarged second image in response to a second triggering operation for the enlarged second image.

9. The system of claim 5, wherein the report generating device is further configured to delete quantified parameter information corresponding to a target second ROI in the human readable report in response to a region deletion operation for the target second ROI in the second image in the human readable report.

10. The system of claim 1, wherein the report generating device is further configured to:
determine a region identifying model corresponding to the target part; and
input the first image into the region identifying model corresponding to the target part to obtain the first ROI in the first image.

11. A medical image processing method applied in the medical image processing system of claim 1, comprising:
acquiring the first image and the second image of the target part acquired by the medical imaging device, the first image being used for depicting the anatomy structure of the target part, and the second image being used for depicting the quantified parameter information of the target part;
identifying the first ROI in the first image.
performing the registration for the first image and the second image to obtain the second ROI in the second image, the second ROI in the second image being relevant to the first ROI in the first image; and
generating the human readable report of the target part according to the quantified parameter information corresponding to the second ROI in the second image.

12. The method of claim 11, wherein the performing the registration for the first image and the second image to obtain the second ROI in the second image comprises:
performing the registration for the first image and the second image to obtain a mapping relationship between the first image and the second image; and
determining, according to the mapping relationship, an image region in the second image relevant to the first ROI to be the second ROI.

13. The method of claim 12, wherein the determining, according to the mapping relationship, an image region in the second image relevant to the first ROI to be the second ROI comprises:
determining first location information of the first ROI in the first image;
determining, according to the mapping relationship, the second location information in the second image relevant to the first location information; and
determining the second ROI in the second image according to the second location information.

14. The method of claim 11, wherein:
at least one second image is provided, and at least one second ROI in each of the at least one second image is provided;
each of the at least one second image is used for depicting quantified parameter information of the target part in different dimensions;
the method further comprises generating the human readable report of the target part based on the first image, each of the at least one second image, and each quantified parameter information corresponding to each of the at least one second ROI.

15. The method of claim 14, further comprising:
acquiring an information reference value corresponding to each quantified parameter information;
generating an information abnormality marker in a case that the quantified parameter information does not conform to the information reference value; and
generating the human readable report according to the first image, each of the at least one second image, each quantified parameter information, each information reference value, and each information abnormality marker.

16. The method of claim 15, further comprising:
presenting a display window displaying the second image in response to a triggering operation for the state information of the target part in the human readable report, wherein a target second image corresponding to the state information of the target part is shown in the display window displaying the second image.

17. The method of claim 15, further comprising:
determining a third ROI in the target second image in response to a region selection operation for the target second image in a display window displaying the second image;
determining a fourth ROI relevant to the third ROI in each of the at least one second image other than in the target second image; and
updating the human readable report according to quantified parameter information corresponding to the third ROI and quantified parameter information corresponding to the fourth ROI.

18. A computer apparatus, comprising a memory and a processor, wherein, a computer program is stored in the memory, and the processor, when executing the computer program, performs steps of the method of claim 11.

19. A non-transitory computer readable storage medium, having a computer program stored thereon, wherein, the computer program, when executed by a processor, causes the processor to perform steps of the method of claim 11.

20. A computer program product, comprising a computer program, wherein, the computer program, when executed by a processor, causes the processor to perform steps of the method of claim 11.
